(19) United States
(12) Patent Application Publication
(10) Pub. No.: US 2004/0073038 A1
(43) Pub. Date: Apr. 15, 2004

(54) Process of preparing paroxetine and intermediates for use therein

(76) Inventor: George Leo Callewaert, Buckinghamshire (GB)

Correspondence Address: OPPEDAHL AND LARSON LLP, P O BOX 5068, DILLON, CO, 80435-5068, US

(21) Appl. No.: 10/468,865

(22) PCT Filed: Feb. 22, 2002
(86) PCT No.: PCT/GB02/00771

(30) Foreign Application Priority Data
Feb. 24, 2001 (GB) 0104583.0
Oct. 18, 2001 (GB) 0125119.8

(51) Int. Cl. C07D 211/22

(52) U.S. Cl. 546/240

(57) ABSTRACT

A process of preparing paroxetine, a process for preparing intermediates for use in the preparation of paroxetine and specific intermediates useful in paroxetine preparation. The specific intermediates that can be employed include compounds of formulae (V), (VI), or (VII).

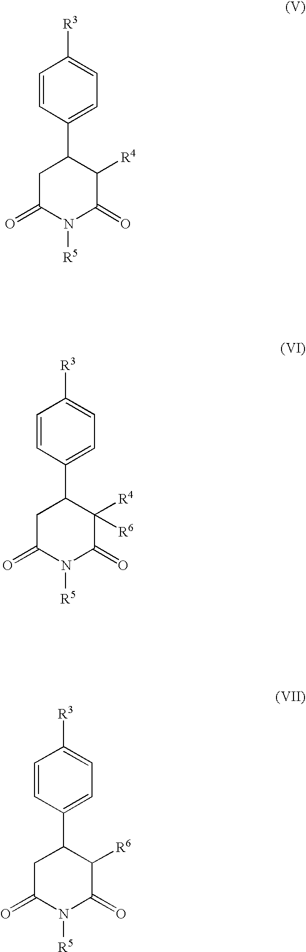

The present invention relates to a process of preparing paroxetine, a process for preparing intermediates for use in the preparation of paroxetine and to specific intermediates useful in paroxetine preparation.

Paroxetine, is a known pharmaceutical compound, for which the systematic name is (3S,4R)-trans-4-(4-fluorophenyl)-3((3,4,methylenedioxyphenyl)oxymethyl) piperidine and has the following general formula (I)

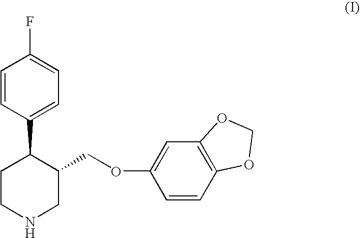

Paroxetine is known to have 5-HT uptake inhibitory activity, and is used in medicine for the treatment of depression and related disorders.

Several methods for the preparation of paroxetine have been proposed in the prior art. Most such methods are, however, unsuitable for industrial use because of their low yields, or the necessity to handle toxic starting materials or intermediates.

Key intermediates have been used in the prior art in the preparation of paroxetine. For example, preferred prior art processes have utilised key intermediates of general formula (II) in the preparation of paroxetine of general formula (I)

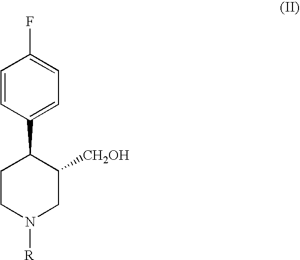

in which R is a protecting group, such as an optionally substituted hydrocarbyl group (for example methyl or benzyl).

Compounds of general formula (II) have been conveniently prepared according to the prior art by the reduction of further key intermediates of formula (III) useful in the preparation of paroxetine of general formula (I)

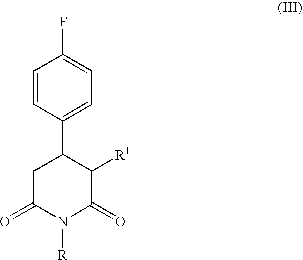

where R is a protecting group substantially as hereinbefore described and R¹ represents —C(═O)OR², where R² can be alkyl as described in, for example, EP-A-223334.

A synthetic route for the preparation of paroxetine of formula (I) utilising key intermediates (II) and (III) can thus be illustrated by the following reaction scheme

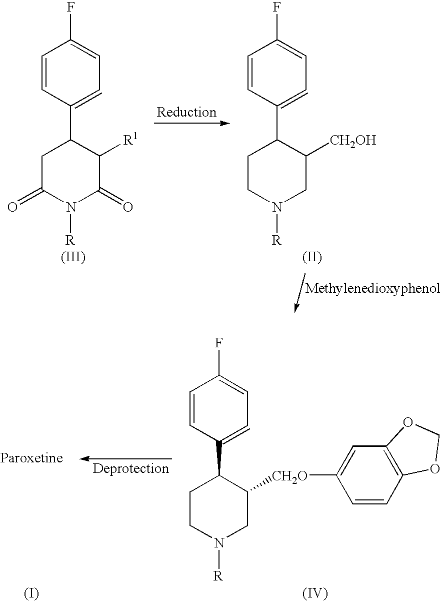

where R and R¹ are substantially as hereinbefore described and a resolution step is carried out prior to coupling of a compound of formula(II) with methylenedioxyphenol.

Preparation of paroxetine of formula (I) employing key intermediate compounds of formulae (II) and (III) substantially as hereinbefore described may be achieved according to a number of different methods known in the prior art. For example, paroxetine of formula (I) can be prepared according to the prior art by coupling of the hydroxyl function of a compound of formula (II) with 3,4 methylenedioxyphenol and subsequent removal of the protecting group R can be readily achieved using known methods. Examples of processes which employ intermediates of formula (II) are disclosed in EP-A-223334, EP-A-374675, EP-A-812827, WO 00/26187 and U.S. Pat. No. 4,007,196.

There are, however, a number of disadvantages associated with the prior art processes of preparing paroxetine of formula (I) and in particular the preparation of key intermediates used therein.

For example, in order to prepare intermediate compounds of is formula (II), the methods of EP-A-812827 and WO 00/26187 require the use of catalytic hydrogenation procedures involving precious metal catalysts. Also, the products of these reactions contain variable amounts of cis/trans isomers.

Furthermore, the processes described in EP-A-223334 use as starting materials mono N substituted amidomalonate esters. These esters are not generally available commercially, and must therefore be made from expensive starting materials using processes with a poor yield. The processes described in EP-A-374675 also require the availability or prior preparation of mono N substituted cinnamides, which cannot readily be prepared in good yields, and often the products are impure.

There is, therefore, a need to provide improved processes for the preparation of paroxetine of formula (I), and novel intermediates useful in the preparation thereof, which alleviate the above mentioned difficulties associated with the prior art processes of preparing paroxetine of formula (I).

According to a first aspect of the present invention, therefore, there is provided a process of preparing a compound of formula (V)

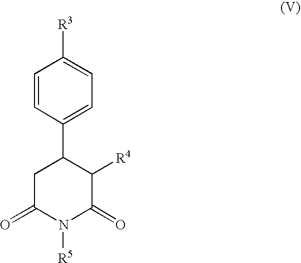

from a compound of formula (VII)

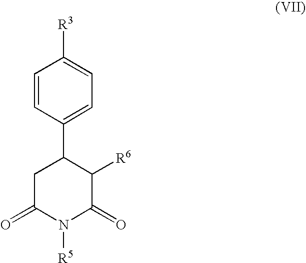

where

R³ represents hydrogen or halo;

R⁴ represents —C(═O)OR⁷, where R⁷ represents optionally substituted alkyl or aralkyl;

R⁵ represents optionally substituted alkyl, alkoxyalkyl, aralkyl or (heterocycle)alkyl; and

R⁶ represents —C(═O)NHR⁵, where R⁵ is as defined above.

More particularly, R³ may represent a halo substituent selected from the group consisting of bromo, chloro, fluoro or iodo, in particular fluoro. Suitably R⁷ as present in compounds of formula (V) represents optionally substituted C₁₋₈ alkyl or benzyl, and can suitably be C₁₋₈alkyl or benzyl, or halo-substituted C₁₋₈alkyl or benzyl, where halo can be selected from the group consisting of bromo, chloro, fluoro or iodo, in particular bromo or chloro, especially chloro. More suitably, R⁷ can be selected from the group consisting of 2-bromoethyl, 2,2,2-trichloroethyl, 2,2,2, -trichloro-1,1-dimethylethyl, t-butyl 2-ethylhexyl and benzyl, and in particular the group consisting of 2,2,2-trichloroethyl, 2,2,2, -trichloro-1,1-dimethylethyl, t-butyl, and benzyl, and more particularly the group consisting of 2,2,2-trichloroethyl, t-butyl and benzyl.

Typically, R⁷ as present in compounds of formula (V) may represent optionally substituted C₁₋₆ alkyl and can suitably be C₁₋₆ alkyl or halo-substituted C₁₋₆ alkyl, where halo can be selected from the group consisting of bromo, chloro, fluoro or iodo, in particular bromo or chloro, especially chloro. Suitably R⁷ can be selected from the group consisting of 2-bromoethyl, 2,2,2-trichloroethyl, 2,2,2-trichloro-1,1-dimethylethyl and t-butyl.

Preferably, R⁵ represents optionally substituted alkyl or aralkyl, such as C₁₋₆ alkyl or (phenyl)C₁₋₆alkyl, and can suitably be selected from the group consisting of methyl, ethyl, n-butyl, benzyl and 4-methylbenzyl, in particular the group consisting of methyl, ethyl, n-butyl and benzyl, and more particularly the group consisting of methyl, ethyl and benzyl.

Typically conversion of a compound of formula (VII) substantially as hereinbefore described to a compound of formula (V) substantially as hereinbefore described comprises at least reacting a compound of formula (VII) with an acylating agent to introduce a R⁴ substituent substantially as hereinbefore described to a phenyl-piperidine compound substantially as herein described. Typically an acylating agent employed in a process according to the present invention can be a chloroformic ester (for example to yield a compound of formula (V) wherein R⁷represents 2,2,2-trichloroethyl, 2,2,2-trichloro-1,1-dimethylethyl or benzyl, especially 2,2,2-trichloroethyl or benzyl) or an organic dicarbonate (for example to yield a compound of formula (V) wherein R⁷ represents t-butyl).

Conveniently, where an acylating agent substantially as hereinbefore described can comprise a chloroformic ester (for example 2,2,2-trichloro-1,1-dimethylethyl chloroformate, 2,2,2-trichloroethyl chloroformate, benzyl chloroformate or the like, especially 2,2,2-trichloroethyl chloroformate, benzyl chloroformate or the like) a compound of formula (VII) may be converted into an alkali metal salt thereof as represented by formula (VIIa) by treatment with a suitable alkali metal base (such as an alkali metal hydride, such as sodium hydride or the like, or an alkali metal alkoxide, such as potassium t-butoxide or the like, or an alkali metal hydroxide or the like) prior to reaction with such a chloroformic ester substantially as hereinbefore described

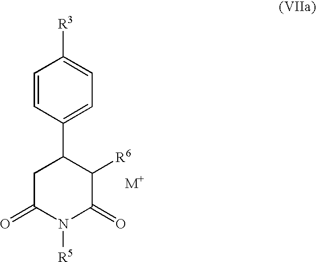

where R³, R⁵ and R⁶ are substantially as hereinbefore described and M represents an alkali metal, such as potassium, sodium or the like.

Preferably reaction with an acylating agent is conducted in the presence of an amine catalyst, for example 4-dimethylamino pyridine (DMAP) or the like. When the acylating agent is an organic dicarbonate, a compound of formula (VII) may be reacted as such without prior conversion to an alkali metal salt.

The above described amide group as represented by R⁶ in a compound of formula (VII) can typically be displaced, such as by treatment with a selected base, such as an alkali metal alkoxide, such as sodium methoxide or the like, or an alkali metal hydroxide, such as potassium hydroxide or the like, to yield a compound of formula (V) substantially as hereinbefore described.

According to a second aspect of the present invention, there is provided a process of preparing a compound of formula (V) from a compound of formula (VI)

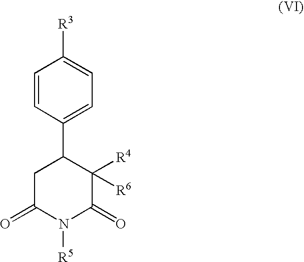

where R³, R⁴, R⁵ and R⁶ (and preferred substituents represented thereby) are substantially as hereinbefore described, typically by displacement of substituent R⁶ by treatment with a suitable base again substantially as hereinbefore described.

A compound of formula (V) as prepared from a compound of formula (VI) typically by treatment with a suitable base, such as an alkali metal base, typically an alkali metal alkoxide, can initially be provided in the form of an alkali metal salt thereof as represented by formula (Va)

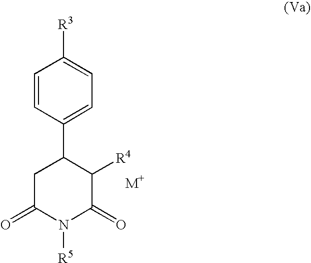

where R³, R⁴ and R⁵ are substantially as hereinbefore described and M represents an alkali metal, such as potassium, sodium or the like. Treatment of a compound of formula (Va) with water or a suitable proton acid can subsequently yield a compound of formula (V) substantially as hereinbefore described.

Preferably a compound of formula (V) is prepared from a compound of formula (VII) via an intermediate compound of formula (VI), each as substantially hereinbefore described. A compound of formula (VI) is therefore typically prepared from a compound of formula (VII), suitably by reaction of the latter with an acylating agent substantially as hereinbefore described.

Although a compound of the general formula (VI) may be readily prepared as a separate pure crystalline intermediate substantially as hereinbefore described, advantageously reactions with the acylating agent and selected base may be carried out sequentially in a ‘one pot’ reaction. This can result in a higher overall yield of isolated compound of general formula (V), with a much simplified procedure.

Preferably a compound of formula (VII) substantially as hereinbefore described is prepared from known starting materials, or starting materials that can be readily prepared from commercially available materials by known synthetic techniques, such as a 4-halocinnamate ester (in particular a 4-fluorocinnamate ester) and a symmetrical N,N′-disubstituted malonamide as represented by general formula (VIII)

HR⁵NC(═O)CH₂C(═O)NR⁵H (VIII)

where R⁵ is substantially as hereinbefore described.

Typical conditions, for example, for the reaction of the above mentioned malonamides, and 4-halocinnamate ester (in particular a 4-fluorocinnamate ester) are in an organic solvent and in the presence of an organic base. In an organic solvent, such as tetrahydrofuran or the like, and in the presence of a base, such as an alkali metal alkoxide, for example potassium t-butoxide or the like, yields and recoveries of compounds of the general formula (VII) may be virtually quantitative. The malonamide and cinnamate starting materials are generally readily available from inexpensive commercially available materials. Furthermore, the cinnamate ester may be prepared in situ from 4-halobenzaldehyde (in particular 4-fluorobenzaldehyde) and the corresponding acetic ester (for example ethyl acetate) in the presence of an organic base (for example sodium methoxide).

Intermediate compounds of general formula (V) substantially as hereinbefore described can readily be reduced by methods well known in the art (for example with metal hydrides, such as lithium aluminium hydride or the like, or with boron hydrides, such as diborane or the like) to give the required intermediate compounds of general formula (IX)

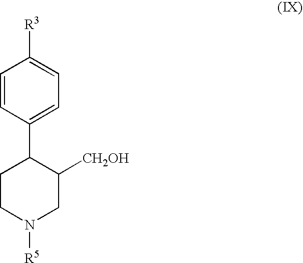

which in turn may then readily be converted into paroxetine of formula (I) substantially as hereinbefore described.

Also, compounds of general formula (V) substantially as hereinbefore described, for example where R⁷ represents t-butyl, may readily be converted to 3-carboxyl derivatives by treatment with trifluoracetic acid or other reagents known to convert tertiary butyl esters into carboxylic acids. Similarly, compounds. of general formula (V) substantially as hereinbefore described for example where R⁷ represents 2,2,2-trichloro-1,1-dimethylethyl or 2,2,2-trichloroethyl, may readily be converted to 3-carboxyl derivatives by treatment with zinc in acetic acid or other reagents known to convert these esters into carboxylic acids. In turn, such carboxylic acids may readily be converted into compounds of general formula (IX) substantially as hereinbefore described or, by choice of a suitable reducing agent, into substituted piperidine carboxylic acid derivatives.

Advantageously, it has been found that compounds of general formula (V) prepared according to the present invention may be formed substantially exclusively as the trans isomers. In turn, the derived compounds of formula (IX) may be also formed substantially exclusively as the desired trans isomers.

The trans isomers of compounds of formula (IX) may be resolved using resolving agents (for example (−)ditoluoyl tartaric acid or the like) according to methods well known in the literature.

Such resolved trans isomers of intermediate compounds of formula (IX) substantially as hereinbefore described may then be coupled to 3,4 methylenedioxyphenol, by activation of the hydroxyl group, typically as a sulphonic ester or the like (for example with methane sulphonyl chloride or benzene sulphonyl chloride) and subsequent treatment with 3,4-methylenedioxyphenoxide. Removal of the protective group R⁵ can then yield paroxetine of formula (I). Methods for removal of protective groups of these types are well known in the literature, and include for example, treatment with a chloroformic ester (for example 1-chloroethyl chloroformate, 2,2,2-trichloroethyl chloroformate or phenyl chloroformate) and subsequent decomposition of the thus formed carbamate. Alternatively, for example where R⁵ represents benzyl or substituted benzyl, R⁵ can be removed by catalytic hydrogenolysis.

There is therefore, further provided by the present invention a process of preparing paroxetine of formula (I) from an intermediate compound of formula (V) substantially as hereinbefore described, which intermediate compound of formula (V) is prepared from either an intermediate compound of formula (VI) or an intermediate compound of formula (VII) substantially as hereinbefore described. A compound of formula (V) can be reduced to yield a compound of formula (IX), followed by resolution and reaction with methylenedioxyphenol and deprotection to yield paroxetine of formula (I) substantially as hereinbefore described. There is still further provided by the present invention a process of preparing paroxetine of formula (I) from either an intermediate compound of formula (VI) or an intermediate compound of formula (VII) substantially as hereinbefore described.

There are also provided by the present invention novel intermediates for use in the preparation of paroxetine of formula (I). Certain intermediate compounds of formula (V) represent novel intermediates according to the present invention and there is, therefore, provided by a further aspect of the present invention an intermediate compound represented by formula (Vb), and salts thereof substantially as hereinbefore described

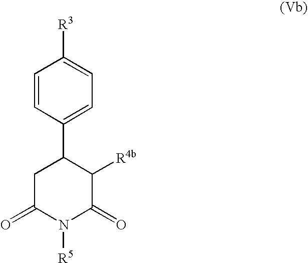

where

R³ and R⁵ are substantially as hereinbefore described; and

R^{4b} represents —C(═O)OR^{4a}, where R^{4a} is selected from the group consisting of 2-bromoethyl, 2,2,2-trichloroethyl, 2,2,2-trichloro-1,1-dimethylethyl, t-butyl, 2-ethylhexyl and benzyl, and in particular is selected from the group consisting of 2,2,2-trichloroethyl, 2,2,2-trichloro-l,1-dimethylethyl, t-butyl and benzyl and more particularly selected from the group consisting of 2,2,2-trichloroethyl, t-butyl and benzyl.

Specific novel intermediates of formula (Vb) are as follows

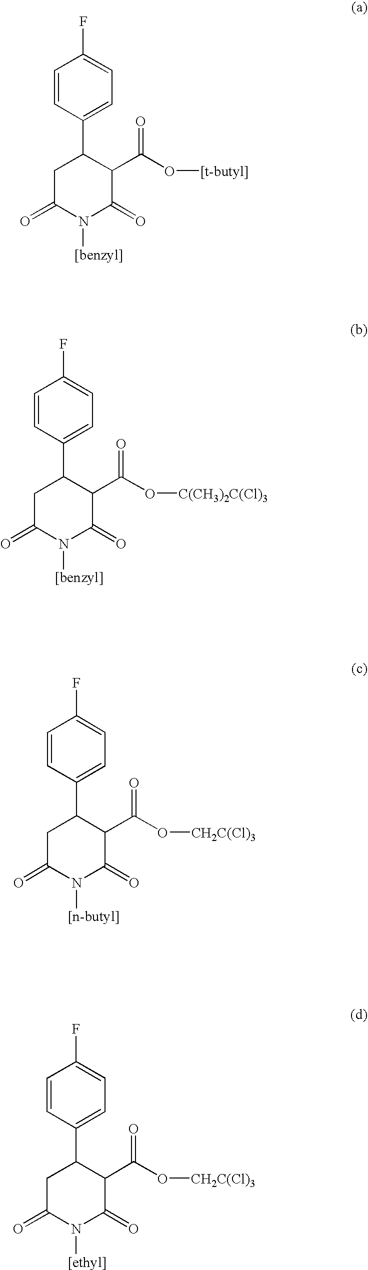

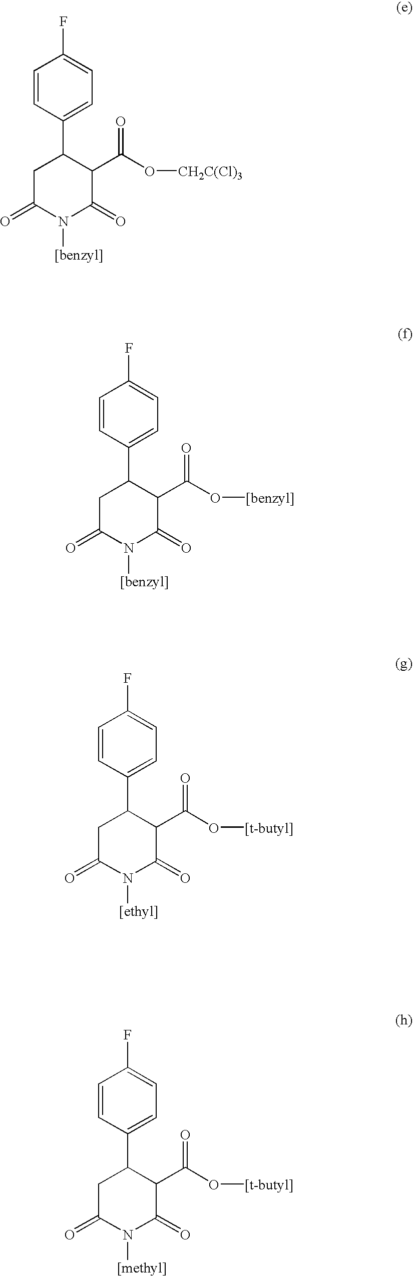

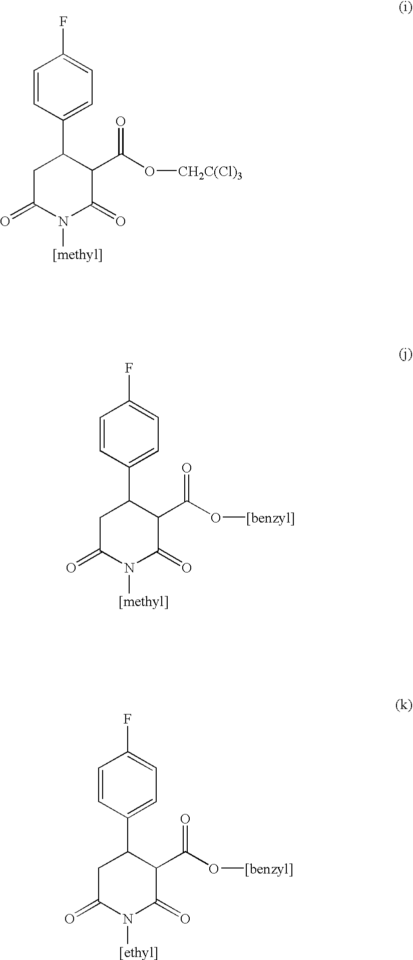

Preferred specific intermediates of formula (Vb) are compounds (a) and (e) to (k) as shown above and falling within the scope of formula (Vb).

There is, therefore, further provided by the present invention a process of preparing paroxetine of formula (I) from an intermediate compound of formula (Vb) substantially as hereinbefore described, and more particularly from any of the above mentioned specific intermediates of formula (Vb) according to the present invention.

The present invention further provides use of a compound of formula (Vb) substantially as hereinbefore described as an intermediate in the preparation of paroxetine of formula (I).

According to a further aspect of the present invention there is also provided an intermediate compound of formula (VII), and salts thereof substantially as hereinbefore described

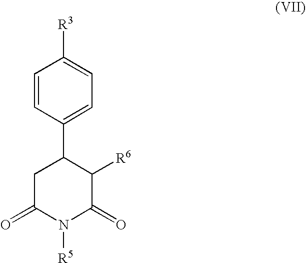

where

R³ represents hydrogen or halo;

R⁵ represents optionally substituted alkyl,

alkoxyalkyl, aralkyl or (heterocycle)alkyl; and

R⁶ represents —C(═O)NHR⁵, where R⁵ is as defined above.

More particularly, R³ may represent a halo substituent selected from the group consisting of bromo, chloro, fluoro or iodo, in particular fluoro.

Preferably, R⁵ represents optionally substituted alkyl or aralkyl, such as C₁₋₆ alkyl or (phenyl)C₁₋₆alkyl, and can suitably be selected from the group consisting of methyl, ethyl, n-butyl, benzyl and 4-methylbenzyl, in particular the group consisting of methyl, ethyl, n-butyl and benzyl, and more particularly the group consisting of methyl, ethyl and benzyl.

Specific novel intermediates of formula (VII) according to the present invention are any one of the following

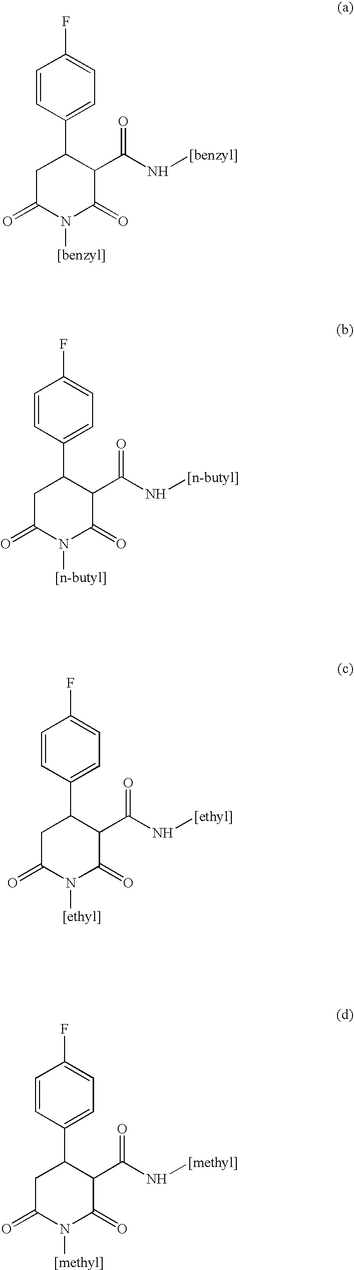

Preferred specific intermediates of formula (VII) are compounds (a), (c) and (d) as shown above and falling within the scope of formula (VII).

There is also provided by the present invention use of a compound of formula (VII) substantially as hereinbefore described as in intermediate in the preparation of paroxetine of formula (I).

According to a still further aspect of the present invention there is provided an intermediate compound of formula (VI)

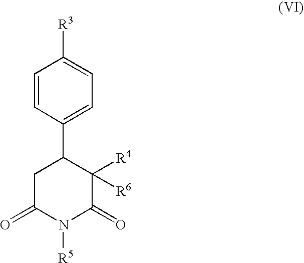

where

R³ represents hydrogen or halo;

R⁴ represents —C(═O)OR⁷, where R⁷ represents optionally substituted alkyl or aralkyl;

R⁵ represents optionally substituted alkyl,

alkoxyalkyl, arallkyl or (heterocycle)alkyl; and

R⁶ represents —C(═O)NHR⁵, where R⁵ is as defined-above.

More particularly, R³ may represent a halo substituent selected from the group consisting of bromo, chloro, fluoro or iodo, in particular fluoro.

Suitably R⁷ as present in compounds of formula (V) represents optionally substituted C₁₋₈ alkyl or benzyl, and can suitably be C₁₋₈alkyl or benzyl, or halo-substituted C₁₋₈alkyl or benzyl, where halo can be selected from the group consisting of bromo, chloro, fluoro or iodo, in particular bromo or chloro, especially chloro. More suitably, R⁷ can be selected from the group consisting of 2-bromoethyl, 2,2,2-trichloroethyl, 2,2,2, -trichloro-1,1-dimethylethyl, t-butyl 2-ethylhexyl and benzyl, and in particular the group consisting of 2,2,2-trichloroethyl, 2,2,2, -trichloro-1,1-dimethylethyl, t-butyl, and benzyl, and more particularly the group consisting of 2,2,2-trichloroethyl, t-butyl and benzyl.

Preferably, R⁵ represents optionally substituted alkyl or aralkyl, such as C₁₋₆ alkyl or (phenyl)C₁₋₆alkyl, and can suitably be selected from the group consisting of methyl,] ethyl, n-butyl, benzyl and 4-methylbenzyl, in particular the group consisting of methyl, ethyl, n-butyl and benzyl, and more particularly the group consisting of methyl, ethyl and benzyl.

Specific novel intermediates of formula (VI) according to the present invention are any one of the following

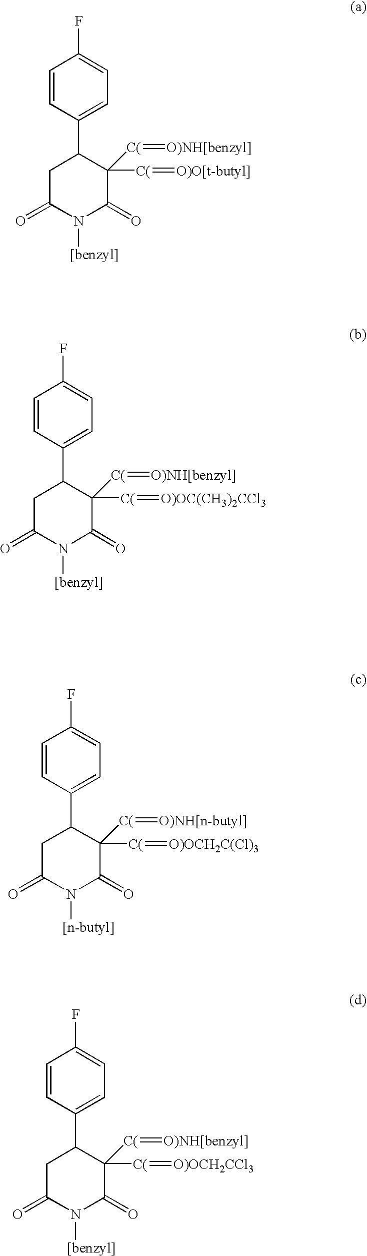

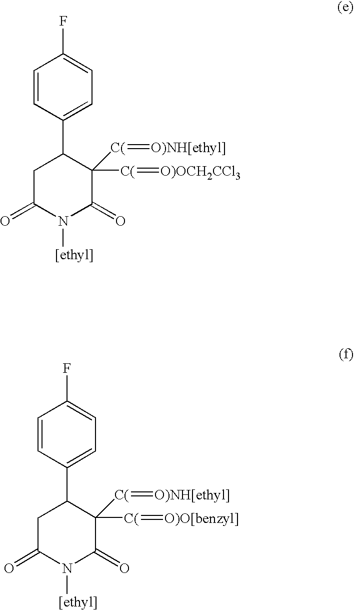

Preferred specific intermediates of formula (VI) are compounds (a), (d), (e) and (f) as shown above and falling within the scope of formula (VI).

There is also still further provided by the present invention use of a compound of formula (VI) substantially as hereinbefore described as an intermediate in the preparation of paroxetine of formula (I).

The present invention will now be further illustrated by the following Examples, which do not limit the scope of the invention in any way.

### EXAMPLE 1

### Preparation of Starting Materials

(i) Trans Ethyl 4-Fluorocinnamate:

This was prepared according to the method described for the preparation of methyl 4-fluorocinnamate in U.S. Pat. No. 5,258,517, but replacing methanol with ethanol. Commercially available 4-fluorocinnamic acid (Aldrich Chemical Company) was used without further purification.

(ii) N,N′ Dibenzylmalonamide:

One equivalent of diethylmalonate was refluxed with two equivalents of benzylamine and a catalytic quantity of ammonium chloride for six hours. The displaced ethanol was then removed by distillation. After cooling, the resulting crystalline mass was taken up in boiling ethyl acetate. After cooling the crystalline product was recovered by filtration, washed with ethyl acetate and dried. A second crop of crystals was obtained by evaporative concentration of the crystallisation mother liquors.

By use of a similar method, N, N′ disubstituted malonamides were prepared from methylamine, ethylamine, butylamine, 2-methoxyethylamine, tetrahydrofurfurylamine and 4-methylbenzylamine.

### EXAMPLE 2

### Preparation of a Compound of General Formula (VII)in Which R³ is Fluoro, R⁵ is Benzyl, and R⁶ is —C(═O)NH[benzyl

A mixture of N,N′dibenzylmalonamide as prepared in Example 1 (7.6 g) and potassium tertiarybutoxide (4.5 g) in tetrahydrofuran (60 ml) was stirred at room temperature for 45 minutes. Over a period of 15 minutes was added a solution of ethyl 4-fluorocinnamate (5.2 g) in tetrahydrofuran (20 ml) as prepared in Example 1. The mixture was left to stir at room temperature overnight. The resulting thick slurry was poured into well stirred water (400 ml) containing acetic acid (2.8 g). Initially an oil formed; after ten minutes stirring this turned into a crystalline solid. This mixture was stirred out at 5° C. for two hours. The crystalline product was filtered under suction, washed with water and pulled as dry as possible on the filter. Subsequently the air dried product was dried over silica gel in vacuo. Yield of crude product was 10.9 g (99%). The crude product was purified by slurrying in a hot mixture of ethyl acetate (70 ml) and ethanol (30 ml). The mixture was left to stand at 5° C. overnight prior to filtration and drying in vacuo. Yield of pure product was 9.5 g, (86%).

### EXAMPLE 3

### Preparation of a Compound According to General Formula (VI), in Which R³ is Fluoro, R⁴ is —C(═O)O [t-butyl] and R⁶ is —C(═O)NH[benzyl] and R⁵ is Benzyl

The product of Example 2 (5 g) was suspended with stirring in dichloromethane (50 ml) and the mixture cooled to 5° C. There was then added di tertiarybutyl dicarbonate (3.3 g) and 4-dimethylamino pyridine (50 mg) and the mixture was left to stir at 5° C. overnight. The resulting clear pale yellow/green solution was taken to dryness on a rotary evaporator to leave a semi-solid mass. This product was taken up in hot isopropanol (25 ml) and left to crystallise at 5° C. The resulting thick crystalline slurry was filtered under suction, washed with a little cold isopropanol and dried in vacuo. Yield was 5 g (81%).

### EXAMPLE 4

### Preparation of a Compound of General Formula (V) in Which R³ is Fluoro, R⁴ is —C(═O)O[t-butyl] and R⁵ is Benzyl

The product of Example 3 (5 g) was dissolved in tetrahydrofuran (50 ml). There was then added potassium tertiary butoxide (1.2 g) and ethanol (1.2 g) and the resulting mixture stirred at room temperature for three hours. The resulting fine suspension was then poured into an ice cold mixture of water (200 ml), lN hydrochloric acid (50 ml) and dichloromethane (50 ml) and the mixture stirred vigorously for a few minutes. The organic layer was separated and the remaining aqueous layer was extracted with two 10 ml portions of dichloromethane. The combined dichloromethane extracts were dried by stirring with magnesium sulphate for fifteen minutes. The magnesium sulphate was removed by filtration and, after washing the magnesium sulphate with a little dichloromethane, the combined dichloromethane extracts were concentrated on a rotary evaporator to an oily residue. This residue was taken up in hot isopropanol (20 ml) and left to crystallise at 5° C. The resulting thick crystalline slurry was filtered under suction, washed with a little cold isopropanol and dried in vacuo. Yield was 3 g (80%) as pure trans product.

### EXAMPLE 5

### Preparation of Title Compound of Example 4 of General Formula (V) Direct From Compound of General Formula (VII) (‘one pot’).

The product of Example 2 (2 g) was suspended with good stirring in tetrahydrofuran (20 ml) and the mixture cooled to 5° C. There was then added di tertiarybutyl dicarbonate (1.3 g) and 4-dimethylamino pyridine (20 mg), and the mixture stirred overnight at 5° C. To the resulting pale orange solution was added potassium tertiary butoxide (0.6 g) and ethanol (0.6 g) and the mixture stirred at room temperature for three hours. The mixture was then poured into an ice cold mixture of water (80 ml), 1N hydrochloric acid (20 ml) and dichloromethane (20 ml) and the mixture stirred vigorously for a few minutes. The organic layer was separated and the remaining aqueous layer extracted with two 10 ml portions of dichloromethane. The combined dichloromethane extracts were dried by stirring with magnesium sulphate for fifteen minutes. The magnesium sulphate was removed by filtration and, after washing the magnesium sulphate with a little dichloromethane, the combined dichloromethane extracts were concentrated on a rotary evaporator to an oily residue. This residue was taken up in hot isopropanol (10 ml) and left to crystallise at 5° C. The resulting thick crystalline slurry was filtered under suction, washed with a little cold isopropanol and dried in vacuo. Yield was 1.3 g (70%) as pure trans product.

### EXAMPLE 6

### Preparation of a Compound of General Formula (VII) in Which R³ is Fluoro, R⁵ is Benzyl and R⁶ is —C(═O)NH[benzyl], Direct From 4-fluorobenzaldehyde

A solution of 4-fluorobenzaldehyde (1.3 g)in ethyl acetate (5 ml) was added to a well stirred suspension of sodium methoxide (1.6 g) in ethyl acetate (20 ml) over 20 minutes. The mixture was then stirred for 30 minutes at room temperature. N,N′ dibenzylmaloiamide (2.9 g) was then added and the mixture stirred for 3 hours at room temperature. Water (25 ml) containing dissolved acetic acid (1.8 g) was chilled to 5° C. and then added with good stirring and the mixture stirred for 30 minutes. At this point the mixture consisted of an aqueous phase, a solvent phase and suspended solids. The solvent ethyl acetate was removed under reduced pressure and the solids recovered by filtration. The solid product was then dried, washed with a little ethyl acetate and then dried in vacuo. Yield of product as a white powder 2.7 g 61%.

### EXAMPLE 7

### Preparation of a Compound of General Formula (VI) in Which R³ is Fluoro, R⁴ is —C(═O)O[2.2,2-trichloro-1,1-dimethylethyl], R⁵ is Benzyl, and R⁶ is —C(═O)NH[Benzyl]

A sample of the compound of Example 6 (1.03 g) was suspended with stirring in tetrahydrofuran (20 ml) and sodium hydride as a 60% suspension in mineral oil (0.1 g) was added. A rapid-evolution of hydrogen ensued and a clear solution was obtained. 4-dimethylamino pyridine (0.03 g) was added and the mixture stirred well for 30 minutes. The solution was then chilled to −25° C. and 2,2,2-trichloro-1,1-dimethylethyl chloroformate (0.74 g) was added with good stirring. The resulting mixture was stirred out for 3 hours at 5° C. The mixture was then poured into a mixture of water (100 ml) and dichloromethane (20 ml) with good stirring. The organic layer was separated and washed successively with dilute hydrochloric acid and 1% aqueous sodium bicarbonate solution. The solvent was then removed using a rotary evaporator to the point where the product began to crystallise. Hot isopropanol (10 ml) was added and the resulting clear pale yellow solution was left to cool at 5° C. The white crystalline product which formed was recovered by filtration, washed with a little cold isopropanol and dried in vacuo.

### EXAMPLE 8

### Preparation of a Trans-compound of General Formula (V) in Which R³ is Fluoro, R⁴ is —C(═O)O[2.2.2-trichloro-1,1-dimethylethyl] and R⁵ is Benzyl

A sample of the compound of Example 7 (2.1 g) was added to tetrahydrofuran (20 ml) in which previously had been dissolved potassium tertiary butoxide (0.53 g) and ethanol (0.45 g) and chilled to 5° C. The resulting mixture was stirred at 5° C. for 30 minutes before being poured into a mixture of 0.2N hydrochloric acid (80 ml) and dichloromethane (20 ml). The organic layer was separated and dried over magnesium sulphate. Removal of the solvent by rotary evaporation resulted in a pale yellow oil which was taken up in hot isopropanol (10 ml). This solution was left to cool at 5° C. The white crystalline product which formed was recovered by filtration, washed with a little cold isopropanol and dried in vacuo.

### EXAMPLE 9

### Preparation of a Trans-compound of General Formula (V) in Which R³ is Fluoro, R⁴ is —C(═O)O[2.2.2-trichloroethyl] and R⁵ is n-butyl

A sample of the compound of general formula (VII) in which R⁵ is n-butyl and R⁶ is —C(═O)NH[n-butyl] (prepared using techniques as described in Example 2) (0.9 g) was dissolved in tetrahydrofuran (20 ml) and sodium hydride as a 60% suspension in mineral oil (0.1 g) was added. A rapid evolution of hydrogen ensued and a clear solution was obtained. 4-dimethylamino pyridine (0.03 g) was added and the mixture stirred well for 30 minutes. The solution was chilled to −25° C. and 2,2,2-trichloroethyl chloroformate (0.75 g) was added with good stirring. The resulting mixture was stirred out for 3 hours at 5° C. Sodium methoxide (0.17 g) was added and the mixture was stirred at 5° C. for 30 minutes before being poured into a mixture of 0.2N hydrochloric acid (80 ml) and dichloromethane (20 ml). The organic layer was separated and dried over magnesium sulphate. Removal of the solvent by rotary evaporation resulted in a pale yellow oil which was taken up in hot isopropanol (4 ml). After cooling to 5° C. and scratching with a glass rod a white crystalline product formed which, after standing for 2 hours, was recovered by filtration, washed with a little cold isopropanol and dried in vacuo.

### EXAMPLE 10

### Preparation of a Compound of General Formula (VII) in Which R³ is Fluoro, R⁵ is Ethyl and R⁶ is —C(═O)NH[ethyl], Direct From 4-fluorobenzaldehyde

A solution of 4-fluorobenzaldehyde (2.6 g) in ethyl acetate (10 ml) was added to a well stirred suspension of sodium methoxide (3.2 g) in ethyl acetate (40 ml) dropwise over 30 minutes. The mixture was then stirred for one hour at room temperature. N,N′diethylmalonamide (3.7 g) was then added slowly to the stirred mixture over 30 minutes. The mixture was then stirred for two hours at room temperature after which it was chilled to 5° C. This mixture was then poured into water (50 ml) at 5° C. containing acetic acid (3.6 g) and the whole stirred well for 2-3 minutes. After separation, the organic phase was washed successively with two 20 ml portions of 2% sodium bicarbonate solution, one 20 ml portion of water and one 20 ml portion of saturated sodium chloride solution. The solvent was then removed on a rotary evaporator to leave a pale yellow solid (6.3 g). Recrystallisation of this solid from ethyl acetate—heptane mixture resulted in a white crystalline product (5.3 g).

In an entirely analogous procedure starting from 4-fluorobenzaldehyde (2.6 g) and NN′dimethyl malonamide (3.1 g) a compound of general formula (VII) in which R⁵ is methyl and R⁶ is —C(═O)NH(methyl) was prepared.

### EXAMPLE 11

### Preparation of a Compound of General Formula (V) in Which R³ is Fluoro, R⁴ is —C(═O)O(Benzyl) and R⁵ is Ethyl

A sample of the title compound of Example 10(1.0 g) was dissolved in tetrahydrofuran (10 ml) and 4-dimethylamino pyridine (0.04 g) added and stirred to dissolve. After chilling this solution to −20° C. potassium t-butoxide (0.4 g) was added and stirred to dissolve. The solution was then chilled back to −20° C. and benzyl chloroformate (0.67 g) was added with good stirring. The mixture was then left to stir overnight at 5° C. Sodium methoxide (0.25 g) was then added and the mixture left to stir at room temperature for 2 hours. The mixture was then poured into a mixture of water (100 ml), concentrated hydrochloric acid (2 ml) and dichloromethane (20 ml) with vigorous stirring. The organic phase was separated and the remaining aqueous phase washed with dichloromethane (10 ml). The combined organic extracts were dried over magnesium sulphate (1.5 g) and then concentrated to a cherry coloured oil on a rotary evaporator. This oil was taken up in hot isopropanol (5 ml) and left overnight at 5° C. when crystallisation occurred. Filtration of the crystals, followed by washing with fresh cold isopropanol, resulted in a white crystalline product.

### EXAMPLE 12

### Preparation of a Compound of General Formula (V) in Which R³ is Fluoro, R⁴ is —C(═O)O (2,2,2-trichloroethyl) and R⁵ is Ethyl.

A sample of the title compound of Example 10(1.0 g) was dissolved in ethyl acetate (10 ml) and dimethylamino pyridine (0.04 g) added and stirred to dissolve. After chilling this solution to −20° C. a previously chilled 50% w/w aqueous solution of potassium hydroxide (10 g) was added. 2,2,2-trichloroethyl chloroformate (0.85 g) was then added and the mixture stirred gently for 1 hour while allowing the temperature to rise to 4° C. After separation of the potassium hydroxide solution, the ethyl acetate solution was washed successively with 5% w/v acetic acid solution (20 ml), water (10 ml) and brine (10 ml). Evaporation of the ethyl acetate solution on a rotary evaporator left a cloudy pale yellow oil. Addition of isopropanol (3 ml) and warming gave a clear pale yellow solution which on cooling deposited white crystals. The white crystalline product was recovered by filtration, washed with a little cold isopropanol and dried in vacuo.

In a second experiment a solution of a compound of general formula (VII) in which R⁵ is ethyl and R⁶ is —C(═O)NH(ethyl) in ethyl acetate as prepared in Example 10 prior to evaporation was treated similarly to give the same result.

### EXAMPLE 13

### Preparation of a Trans-compound of General Formula (IX) in Which R³ is Fluoro and R⁵ is n-butyl

A sample of a compound of general formula (V) in which R³ is fluoro, R⁵ is n-butyl and R⁴ is —C(═O)O[2,2,2-trichloroethyl] (1.0 g) as prepared in Example 9 was dissolved in tetrahydrofuran (10 ml) and the solution chilled to −20° C. There was then added lithium aluminium hydride 1.0M solution in tetrahydrofuran (10 ml). The resulting warm solution was cooled to 5° C. and left to stir overnight at this temperature. Thereafter it was stirred out at room temperature for three hours. Water (6 ml) was added extremely slowly with good stirring and applied cooling followed by 0.25M sodium hydroxide solution (4 ml). The mixture was then stirred for 30 minutes at room temperature. The mixture was then filtered followed by a wash through with ethyl acetate (50 ml). The organic phase was separated and dried with magnesium sulphate. After removal of the magnesium sulphate the solvent was removed on a rotary evaporator to leave an almost colourless oily residue. Trituration with a small quantity of diisopropyl ether resulted in the formation of a white solid. Recrystallisation of this solid from cyclohexane resulted in a white crystalline product.

In entirely analagous procedures, a compound of general formula (V) in which R³ is fluoro, R⁵ is ethyl and R⁴ is C(═O)O[2,2,2-trichloroethyl] was converted to a compound of general formula (IX) in which R⁵ is ethyl and a compound of general formula (V) in which R³ is fluoro, R⁵ is benzyl and R⁴ is —C(═O)O[2,2,2-trichloroethyl] was converted to a compound of general formula (IX) in which R⁵ is benzyl.

### EXAMPLE 14

### Resolution of a Trans Compound of General Formula (IX) in Which R³ is Fluoro and R⁵ is Ethyl

(i) Preparation of Seed Crystals

A sample of a compound of general formula (IX) in which R³ is fluoro and R⁵ is ethyl (100 mg) was dissolved in acetone (1.0 ml) with gentle warming. Di-p-toluoyl-L-tartaric acid (165 mg) was dissolved in acetone (1.0 ml) and the two solutions were mixed well. No crystallisation occurred. A few drops of this mixture were taken and allowed to evaporate to leave a cloudy oily residue. Trituration of this residue with cyclohexane resulted in the formation of crystals. These crystals were added back to the solution and after gentle mixing for a few minutes a general crystallisation occurred.

(ii) Resolution

A sample of a compound of general formula (IX) in which R³ is fluoro and R⁵ is ethyl (4.8 g) was dissolved in acetone containing 5% v/v water (50 ml) with warming to 40° C. Di-p-toluoyl-L-tartaric acid (10 g) was dissolved in acetone (50 ml) and the solution warmed to 40° C. The above two solutions were mixed and gently stirred at 40° C. A sample of seed crystals prepared as above was added and the mixture gently stirred allowing the temperature to gradually fall to room temperature over a period of one hour. Thereafter it was cooled with stirring to 5° C. and stirred at this temperature for one hour. The deposited crystals were recovered by filtration, washed with a little fresh acetone and dried in vacuo.

(iii) (−)trans 4-(4′-fluorophenyl)-3-hydroxymethyl-1-ethylpineridine

A sample of the above salt (1.5 g) was stirred in water (10 ml) and the pH adjusted to 11.2 with added 2N sodium hydroxide solution. Dichloromethane (10 ml) was then added and after vigorous mixing the dichloromethane phase was separated. The remaining aqueous layer was extracted with a further aliquot of dichloromethane (10 ml). The dichloromethane extracts were combined and the dichloromethane removed by evaporation to leave an oily residue. This residue was taken up in hot isopropyl ether (10 ml) which on cooling and with scratching resulted in the formation of crystals of the title compound. The crystals were recovered by filtration, washed with isopropyl ether and dried in vacuo.

### EXAMPLE 15

### Preparation of Paroxetine Hydrochloride

(i) Preparation of (−)trans-4-(4′-fluorophenyl)-3-(3,4-methylenedioxyphenoxymethyl)-1-ethylpiperidine

(−) trans-4-(4′-fluorophenyl)-3-hydroxymethyl-1-ethylpiperidine (as prepared in Example 14 part (iii)) (10 g) was dissolved in toluene (60 ml) together with triethylamine (10 ml). With stirring at room temperature a solution of methane sulphonyl chloride (5.8 g) in toluene (10 ml) was added dropwise over 1 hour and the resulting mixture stirred at room temperature for 3 hours. The toluene solution was washed with water (60 ml) and concentrated to an oil on a rotary evaporator. The oil was taken up in fresh toluene (60 ml) and treated sequentially with a solution of 3,4-methylenedioxyphenol (6.4 g) in isobutylmethyl carbinol (25 ml) and a solution of sodium hydroxide (2.1 g) in water (5 ml). The resulting mixture was refluxed for 3 hours. When cool, the organic mixture was washed with three portions of water (30 ml) and the combined water washes were extracted with toluene (60 ml) which was combined with the organic phase. The organic phase was concentrated under reduced pressure to leave an oil which was taken up in acetone (50 ml). Dropwise addition of concentrated hydrochloric acid caused the product to crystallise as the hydrochloride salt which was filtered, washed with acetone and dried in vacuo.

(ii) Preparation of Paroxetine Hydrochloride

(−) trans-4-(4′-fluorophenyl)-3-(3,4-methylenedioxyphenoxymethyl)-1-ethylpiperidine hydrochloride (10 g) was added to a mixture of toluene (70 ml) and water (35 ml) and with vigorous stirring sodium hydroxide solution (4N) was added to pH 12. The toluene phase was separated and the aqueous phase extracted with a further portion of toluene (35 ml). The combined toluene extracts were dried over potassium carbonate following which the toluene was removed on a rotary evaporator to leave an oil which was taken up in dichloromethane (50 ml) and the solution cooled to 0° C. A solution of phenyl chloroformate (7.9 g) in dichloromethane (10 ml) was added dropwise with good stirring over 20 minutes and the resulting clear solution was held at room temperature for 3 hours. The solution was then washed successively with 1N sodium hydroxide solution (50 ml) and two portions of 6N hydrochloric acid (50 ml). The organic phase was concentrated on a rotary evaporator to leave an oil which was taken up in toluene (70 ml). After addition of potassium hydroxide (6.5 g) the mixture was heated to reflux for 2 hours. Water (50 ml) was added, the organic phase separated and the aqueous phase extracted with two portions of toluene (30 ml). The organic phase and the toluene extracts were combined and concentrated to an oil on a rotary evaporator. The oil was taken up in isopropanol (30 ml) and concentrated hydrochloric acid was added dropwise to cause crystallisation of paroxetine hydrochloride. The crystalline product was filtered, washed with isopropanol and dried in vacuo. Physical and spectroscopic characteristics of the product were in accordance with published data for paroxetine hydrochloride.

1. A process of preparing a compound of formula (V) 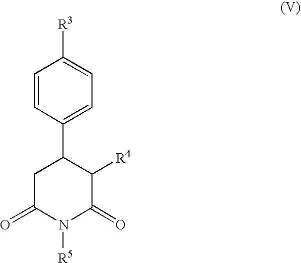
from a compound of formula (VII) 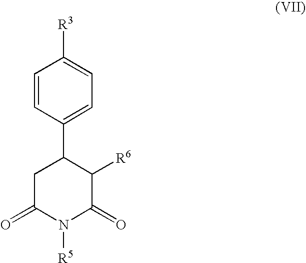
where
R³ represents hydrogen or halo;
R⁴ represents —C(═O)OR⁷where R⁷ represents optionally substituted alkyl or aralkyl;
R⁵ represents optionally substituted alkyl, alkoxyalkyl, aralkyl or (heterocycle)alkyl; and
R⁶ represents —C(═O)NHR⁵, where R⁵ is as defined above.

2. A process according to claim 1, wherein R³ represents a halo substituent selected from the group consisting of bromo, chloro, fluoro and iodo.

3. A process according to claim 2, wherein R³ represents fluoro.

4. A process according to any of claims 1 to 3, wherein R⁷ represents C₁₋₈alkyl, benzyl, halo-substituted C₁₋₈alkyl or halo-substituted benzyl, where halo is selected from the group consisting-of bromo, chloro, fluoro and iodo.

5. A process according to claim 4, wherein R⁷ is selected from the group consisting of 2-bromoethyl, 2,2,2-trichloroethyl, 2,2,2, -trichloro-l,1-dimethylethyl, t-butyl, 2-ethylhexyl and benzyl.

6. A process according to claim 5, wherein R⁷ is selected from the group consisting of 2,2,2-trichloroethyl, t-butyl, and benzyl.

7. A process according to any of claims 1 to 6, wherein R⁵ represents optionally substituted alkyl or aralkyl.

8. A process according to claim 7, wherein R⁵ represents optionally substituted C₁₋₆alkyl or (phenyl)C₁₋₆alkyl.

9. A process according to claim 8, wherein R⁵ is selected from the group consisting of methyl, ethyl, n-butyl, benzyl and 4-methylbenzyl.

10. A process according to claim 9, wherein R⁵ is selected from the group consisting of methyl, ethyl and benzyl.

11. A process according to any of claims 1 to 10, wherein conversion of a compound of formula (VII) to a compound of formula (V) comprises at least reacting a compound of formula (VII) with an acylating agent.

12. A process according to claim 11, wherein the acylating agent is a chloroformic ester or an organic dicarbonate.

13. A process according to claim 12, wherein the acylating agent comprises a chloroformic ester and a compound of formula (VII) is converted into an alkali metal salt thereof as represented by formula (VIIa), by treatment with an alkali metal base prior to reaction with the chloroformic ester 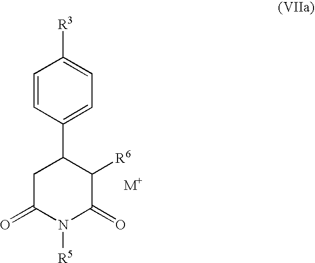
where R³, R⁵ and R⁶ are as defined in any of claims 1 to 12 and M represents an alkali metal.

14. A process of preparing a compound of formula (V) 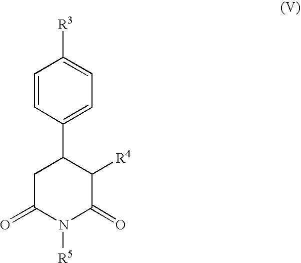
from a compound of formula (VI) 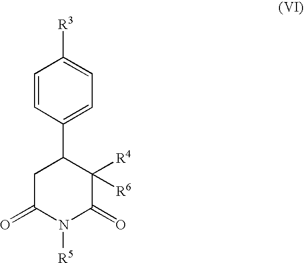
where
R³ represents hydrogen or halo;
R⁴ represents —C(═O)OR⁷₁, where R⁷ represents optionally substituted alkyl or aralkyl;
R⁵ represents optionally substituted alkyl, alkoxyalkyl, aralkyl or (heterocycle)alkyl; and
R⁶ represents —C(═O)NHR⁵, where R⁵ is as defined above.

15. A process according to claim 14, wherein R³ represents a halo substituent selected from the group consisting of bromo, chloro, fluoro and iodo.

16. A process according to claim 15, wherein R³ represents fluoro.

17. A process according to any of claims 14 to 16, wherein
R⁷ represents C₁₋₈alkyl, benzyl, halo-substituted C₁₋₈alkyl or halo-substituted benzyl, where halo is selected from the group consisting of bromo, chloro, fluoro and iodo.

18. A process according to claim 17, wherein R⁷ is selected from the group consisting of 2-bromoethyl, 2,2,2-trichloroethyl, 2,2,2, -trichloro-l,1-dimethylethyl, t-butyl, 2-ethylhexyl and benzyl.

19. A process according to claim 18, wherein R⁷ is selected from the group consisting of 2,2,2-trichloroethyl, t-butyl and benzyl.

20. A process according to any of claims 14 to 19, wherein
R⁵ represents optionally substituted alkyl or aralkyl.

21. A process according to claim 20, wherein R⁵ represents optionally substituted C₁₋₆alkyl or (phenyl)C₁₋₆alkyl.

22. A process according to claim 21, wherein R⁵ is selected from the group consisting of methyl, ethyl, n-butyl, benzyl and 4-methylbenzyl.

23. A process according to claim 22, wherein R⁵ is selected from the group consisting of methyl, ethyl and benzyl.

24. A process according to any of claims 14 to 23, wherein
a compound of formula (V) is prepared from a compound of formula (VI) by treatment with an alkali metal base so as to initially provide a compound of formula (V) in the form of an alkali metal salt thereof as represented by formula (Va) 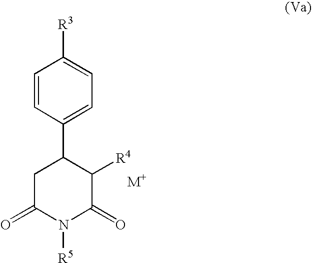
where R³, R⁴ and R⁵ are as defined in any of claims 14 to 23 and M represents an alkali metal.

25. A process according to any of claims 1 to 13, wherein
a compound of formula (V) is prepared from a compound of formula (VII) via an intermediate compound of formula (VI) as defined in any of claims 14 to 24.

26. A process according to any of claims 1 to 13 or 25, wherein a compound of formula (VII) is prepared from a 4-halocinnamate ester and a symmetrical N,N′-disubstituted malonamide as represented by general formula (VIII)
HR⁵NC(═O)CH₂C(═O)NR⁵H (VIII)
where R⁵ is as defined in any of claims 1 to 13.

27. A process of preparing paroxetine of formula (I) 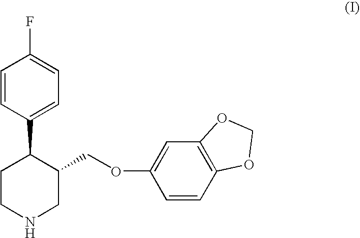
from an intermediate compound of formula (V), which intermediate compound of formula (V) is prepared from either an intermediate compound of formula (VI) or an intermediate compound of formula (VII) according to any of claims 1 to 26.

28. A compound of formula (Vb), and salts thereof 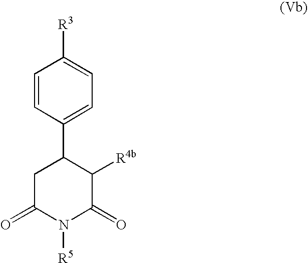
where
R³ represents hydrogen or halo;
R^{4b} represents —C(═O)OR^{4a}, where R^{4a} is selected from the group consisting of 2-bromoethyl, 2,2,2-trichloroethyl, 2,2,2-trichloro-1,1-dimethylethyl and t-butyl, 2-ethylhexyl and benzyl; and
R⁵ represents optionally substituted alkyl, alkoxyalkyl, aralkyl or (heterocycle)alkyl.

29. A compound according to claim 28, wherein R^{4b} is selected from the group consisting of 2,2,2-trichloroethyl, t-butyl and benzyl.

30. A compound according to claim 28 or 29, wherein R³ represents a halo substituent selected from the group consisting of bromo, chloro, fluoro and iodo.

31. A compound according to claim 30, wherein R³ represents fluoro.

32. A compound according to any of claims 28 to 31, wherein R⁵ represents optionally substituted alkyl or aralkyl.

33. A compound according to claim 32, wherein R⁵ represents optionally substituted C₁₋₆alkyl or (phenyl)C₁₋₆alkyl.

34. A compound according to claim 33, wherein R⁵ is selected from the group consisting of methyl, ethyl, n-butyl, benzyl and 4-methylbenzyl.

35. A compound according to claim 34, wherein R⁵ is selected from the group consisting of methyl, ethyl and benzyl.

36.

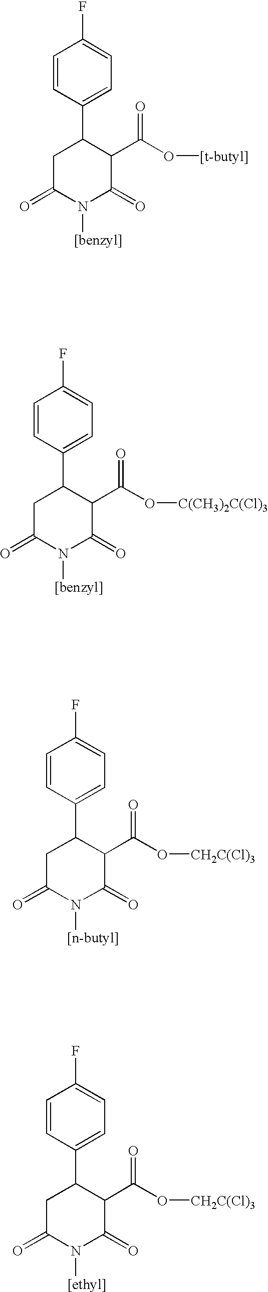

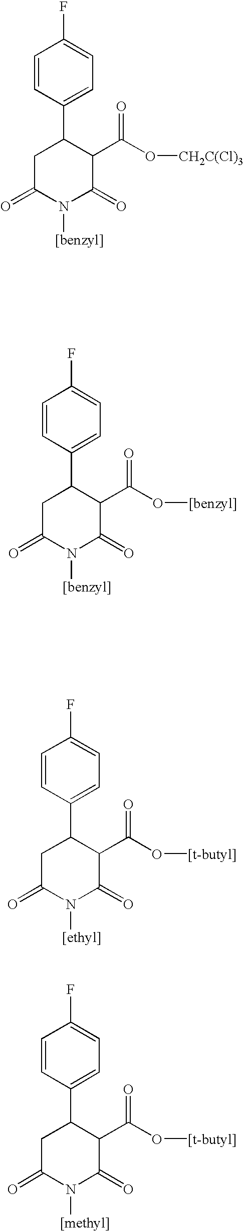

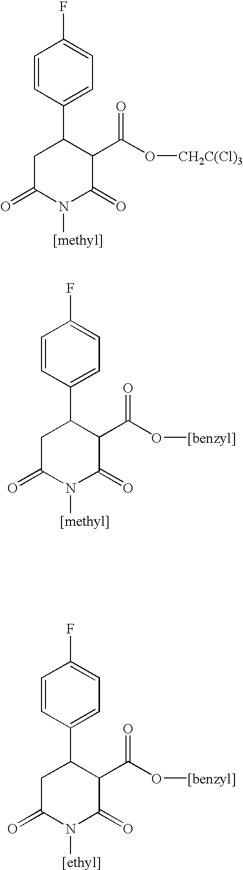

37. A compound of formula (VII), and salts thereof 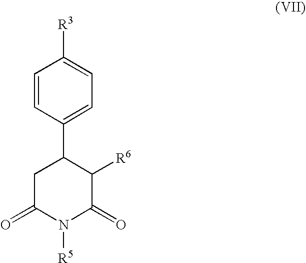
where
R³ represents hydrogen or halo;
R⁵ represents optionally substituted alkyl, alkoxyalkyl, aralkyl or (heterocycle)alkyl; and
R⁶ represents —C(═O)NHR⁵, where R⁵ is as defined above.

38. A compound according to claim 37, wherein R³ represents a halo substituent selected from the group consisting of bromo, chloro, fluoro and iodo.

39. A compound according to claim 38, wherein R³ represents fluoro.

40. A compound according to any of claims 37 to 39,
wherein R⁵ represents optionally substituted alkyl or aralkyl.

41. A compound according to claim 40, wherein R⁵ represents 5 optionally substituted C₁₋₆alkyl or (phenyl)C₁₋₆alkyl.

42. A compound according to claim 41, wherein R⁵ is selected from the group consisting of methyl, ethyl, n-butyl, benzyl and 4-methylbenzyl.

43. A compound according to claim 42, wherein R⁵ is selected from the group consisting of methyl, ethyl and benzyl.

44.

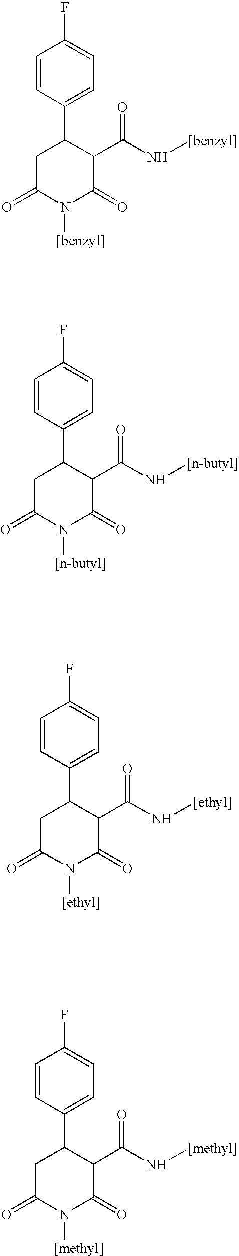

45. A compound of formula (VI) 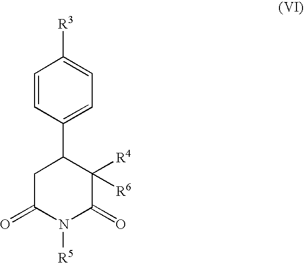
where
R³ represents hydrogen or halo;
R⁴ represents —C(═O)OR⁷, where R⁷ represents optionally substituted alkyl or aralkyl;
R⁵ represents optionally substituted alkyl, alkoxyalkyl, arallkyl or (heterocycle)alkyl; and
R⁶ represents —C(═O)NHR⁵, where R⁵ is as defined above.

46. A compound according to claim 45, wherein R³ represents a halo substituent selected from the group consisting of bromo, chloro, fluoro an iodo.

47. A compound according to claim 46, wherein R³ represents fluoro.

48. A compound according to any of claims 45 to 47, wherein R⁷ represents C₁₋₈alkyl, benzyl, halo-substituted C₁₋₈alkyl or halo-substituted benzyl, where halo is selected from the group consisting of bromo, chloro, fluoro and iodo.

49. A compound according to claim 48, wherein R⁷ is selected from the group consisting of 2-bromoethyl, 2,2,2-trichloroethyl, 2,2,2, -trichloro-1,1-dimethylethyl, t-butyl, 2-ethylhexyl and benzyl.

50. A compound according to claim 49, wherein R⁷ is selected from the group consisting of 2,2,2-trichloroethyl, t-butyl and benzyl.

51. A compound according to any of claims 45 to 50, wherein R⁵ represents optionally substituted alkyl or aralkyl.

52. A compound according to claim 51, wherein R⁵ represents optionally substituted C₁₋₆alkyl or (phenyl)C₁₋₆alkyl.

53. A compound according to claim 52, wherein R⁵ is selected from the group consisting of methyl, ethyl, n-butyl, benzyl and 4-methylbenzyl.

54. A compound according to claim 53, wherein R⁵ is selected from the group consisting of methyl, ethyl and benzyl.

55.

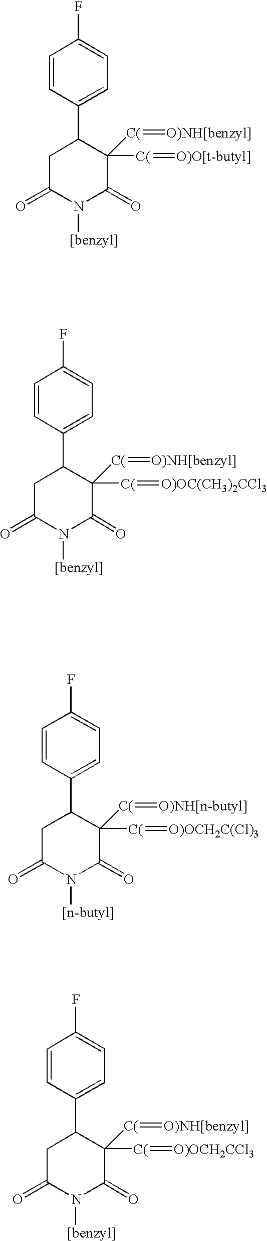

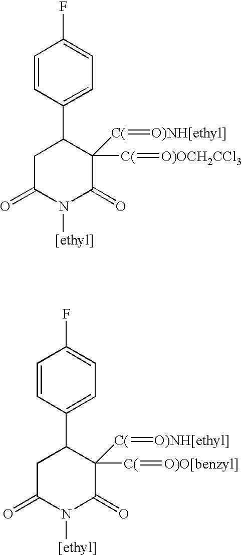

56. A process of preparing paroxetine of formula (I) 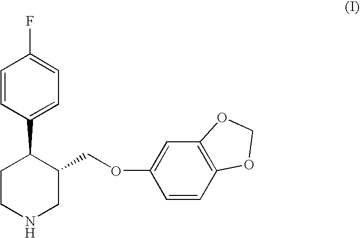
from an intermediate compound as defined in any of claims 28 to 55.

57. Use of an intermediate compound as defined in any of claims 28 to 55, as an intermediate in the preparation of paroxetine of formula (I) [chemical structure]
